(19) United States
(12) Patent Application Publication
(10) Pub. No.: US 2026/0051396 A1
(43) Pub. Date: Feb. 19, 2026

(54) AUTOMATIC CONFIGURATION OF AN OPERATING ROOM USING ANOMALY DETECTION

(71) Applicant: KARL STORZ SE & Co. KG, Tuttlingen (DE)

(72) Inventor: Andreas Klingenberg, Berlin (DE)

(73) Assignee: KARL STORZ SE & Co. KG, Tuttlingen (DE)

(21) Appl. No.: 19/301,259

(22) Filed: Aug. 15, 2025

(30) Foreign Application Priority Data
Aug. 16, 2024 (DE) 10 2024 123 453.9

(51) Int. Cl. G16H 40/20; G16H 40/60

(52) U.S. Cl. CPC G16H 40/20; G16H 40/60

(57) ABSTRACT

Method for the dynamic configuration of an integrated operating room comprising the continuous collection of data relating to device settings, device display, device performance, users, user actions and/or information relating to an ongoing medical intervention, the continuous determination of intervention situations based on the collected data, the comparison of current device settings with device settings in corresponding intervention situations of comparable medical interventions, the determination of deviations between the current device settings and device settings in corresponding intervention situations of comparable medical interventions, and the determination of necessary adjustments to the current device settings in order to attenuate the deviations.

### CROSS-REFERENCE TO RELATED APPLICATIONS

The present application claims the benefit of German Patent Application 102024123453.9 filed on Aug. 16, 2024, titled, “Automatische OP Konfiguration mittels Anomaliedetektion” (“Automatic Configuration of an Operating Room Using Anomaly Detection”), and is incorporated herein by reference in its entirety.

### TECHNICAL FIELD

The present invention relates to automatically configuring an integrated operating room, in particular automatic configuration depending on specific intervention situations.

### BACKGROUND

Modern operating rooms are equipped with a wide variety of equipment. In addition to the actual medical devices, the equipment often includes computers and monitors that control devices, provide staff with information, and record information. Cameras may be utilized as part of a medical intervention or, for example, room cameras may capture and record what is happening in the operating room. Different devices can be partially or fully connected to each other, i.e., networked, and thus exchange data. In one design, operating room devices are networked on an equipment trolley that acts as a control unit for the devices connected to it. In other designs, so-called integrated operating rooms are used, in which a computer is connected to other components or devices to collect and coordinate their data and/or functions.

In such an integrated system, the staff in the operating room not only have access to the patient's medical information, but can also display inter alia device information, such as image data from endoscope cameras, and/or control medical devices and areas of the building services, such as lighting—ideally from a central user interface.

While the integration simplifies operation by the respective staff, the settings themselves must be made by the staff and are based on their wealth of experience and also on their perception. The larger the number of adjustable parameters, the more complex this task becomes. To facilitate this, for example, presets can be defined for certain scenarios so that the user can automatically adjust various parameters of different devices by selecting suitable presets or configurations.

For this, for example, very complex, static configurations of such an integrated operating room can be implemented, wherein these configurations must be selected manually by the user during the intervention and, if necessary, manually adapted to specific situations or changes. However, deviations, for example due to new types of interventions and/or different or new devices used, cannot be covered or taken into account in advance in the static configuration.

In addition to the static nature of the available configurations, the existing options regularly ignore aspects resulting from the combination of different devices. In addition, measured values and further information that are regularly and often collected on a large scale in medical applications are regularly not taken into account, or at least not automatically, when selecting suitable device adaptations.

### SUMMARY

Accordingly, it is an object of the present disclosure to overcome, at least in part, the limitations or deficiencies of the methods known from the prior art and advance the state of the art.

As a general introduction, and in one embodiment, systems and methods are disclosed herein that are directed to the control and management of one or more medical devices (or, more simply, “device” or “devices”), each having at least one configuration or setting that is determinable and/or controllable by another electronic device. The devices are used for medical operations or procedures, such as surgeries, which are embodiments of a “medical intervention” (or, more simply, an “intervention”). Such adjustments or configurations may be determined, in whole or in part, in accordance with the patient's physiology, the procedure being performed, a particular phase of a multi-phase procedure, and/or the physician's needs or preferences. It should be appreciated that devices that do not have adjustments or configurations that are determinable and/or controllable by another electronic device, different from the device itself, are outside the scope of this disclosure.

In another embodiment, a medical intervention comprises the use of one or more devices each having at least one setting. An “intervention situation” defines the particular configurations or settings for such devices for use in a particular situation, such as the patent's physiology, procedure, phase of a multi-phase procedure, and/or physician's needs or preferences. The intervention situation may be provided, such as by a member of the medical staff inputting information into a computer, or automatically determined. For example, a computer determining that an imaging device is being powered up or manipulated may then determine the settings requirements for other devices, such as to power-up and/or configure a display device with the particular input source utilized to receiving images from the imaging device and present the images thereon. Once the settings are determined, the systems and methods then, in one aspect, indicate those settings for application by the medical staff or, in another aspect, automatically configure the other devices without human intervention. It should be appreciated that “intervention situation” refers to those aspects of a medical procedure that will or are reasonably likely to influence the patient's diagnosis, treatment and/or outcome of a medical procedure and excludes aspects that are medically irrelevant, such as administrative or ancillary information, or any information that, if the value of such information were different, would have no influence on the imminent actions being considered or currently being taken by the medical staff performing the medical intervention.

The term, “comparable,” as used herein means identical or substantially identical. For example, “comparable medical intervention,” as used herein, refers to the substantially similar steps taken to perform a first medical intervention as compared to a second medical intervention. The first and second medical interventions may have the same purpose (e.g., remove an appendix) and/or substantially the same steps (e.g., insert an endoscope into the patient's abdomen, find the source of internal bleeding, apply a hemostat, etc.) even if the subject of those steps are not identical (e.g., addressing internal bleeding in the left lumbar region versus addressing internal bleeding in the right iliac region). Variations on specific techniques, to perform the substantially similar steps, may exist due to patient physiology (e.g., body mass index, age, general health), physician's needs or attributes (e.g., left-handed, right-handed, height, fatigue, etc.), and/or physician's preferences (e.g., magnification or illumination level of an instrument, etc.). Similarly, “comparable device settings,” means two devices (or the same device used at different times for comparable medical interventions) has settings that are either identical or substantially (e.g., differences are limited to those that account for differences in patient physiology or physician's needs or preferences).

One aspect is directed to a method for dynamically configuring an integrated operating room, the method comprising the following steps: continuously collecting data relating to device settings, device displays, device performance, users, user actions and/or information relating to an ongoing medical intervention; continuously determining intervention situations based on the collected data; comparing current data with data from a corresponding intervention situation of comparable medical interventions; determining deviations between current data and data from a corresponding intervention situation of comparable medical interventions; and determining necessary adjustments to the current device settings in order to remove or attenuate the deviations. Examples of integrated devices within the meaning of certain aspects are, in particular, medical devices, surgical lighting, documentation systems, monitors, cameras, ventilation systems, air conditioning systems, network components, in particular for receiving and sending data, communication components, as well as various types of sensors and the like. In one embodiment, the steps of determining the intervention situation, comparing data and determining deviations based on the entire acquired data set or on parts of the data set, wherein the individual steps can in particular also relate to different, possibly overlapping areas of the data. In preferred embodiments, individual steps may also be applied sequentially or simultaneously to different, possibly overlapping, areas of the data. For example, in a first step, a deviation from the normal course of a medical intervention, a so-called anomaly, can be detected by comparing the data. In a subsequent step, especially if an anomaly has been detected, individual data areas, such as device settings, can be compared in order to identify possible reasons for the anomaly in the form of deviating device settings. In principle, however, within the meaning of certain aspects of the present disclosure, only device settings can be compared and irregularities can therefore be determined solely on the basis of differing device settings.

In one embodiment, the continuous determination of the intervention situation can be based on user actions, in particular on corresponding user inputs. In this case, user inputs can be captured in particular via a corresponding user interface or via speech recognition from verbal instructions given by the user. Alternatively or in addition, suitable image recognition algorithms can be used to recognize certain actions and/or movement sequences and to derive the respective intervention situation from them.

In one embodiment, the determination of the intervention situation may comprise the comparison of current data with historical data. Preferably, the determination of the current intervention situation can be carried out using an artificial intelligence-based recognition algorithm trained using historical data. Data sets, in particular annotated data sets of historical medical interventions, can be used, preferably using data from a comparable operating room or setup. In particular, when determining the intervention situation, it can be based on the fact that at least some of the data from the same or comparable devices are used as a basis. Preferably, the determination may also include the use of available information relating to the intervention, such as information on the type of medical intervention from a surgical plan or from the patient's medical record. Likewise, the type of operating room or the allocation of the operating room to a specific department can be taken into account in the determination. Accordingly, the allocation of an operating room to an orthopedic department of a hospital or to a dermatological department of a hospital can significantly limit the type of possible medical interventions and thus lead to an improved determination of the intervention situation, since certain intervention situations are only to be expected for certain medical interventions.

In another embodiment, the artificial intelligence-based recognition algorithm comprises or uses a neural network trained for recognition, such as via use of a set of historic medical intervention situations. The training may then comprise collecting the set of historic medical intervention situations, such as from a database, and applying one or more transformations to each medical intervention situation to create a modified set of historic medical intervention situations. The transformations comprising altering a physiological aspect of the patient (e.g., gender, age, body mass, adding other health conditions, subtracting other health conditions), the intervention itself (e.g., adding another intervention, subtracting another intervention, performing the same intervention on a different, but appropriate, part of the body (e.g., left knee versus right knee)), the surgeon (e.g., left handed to right handed or vice versa, adding a visual impairment, removing a visual impairment, surgical device setting preference, level of fatigue, level of skill, etc.). Creating a first training set comprising the collected historic medical intervention situations and the set of modified set of historic medical intervention situations. Training the neural network in a first stage using the first training set. Creating a second training set for a second stage of training comprising the first training set and a set of medical intervention situations incorrectly identified (e.g., wrong procedure) after the first stage of training. Training the neural network in the second stage using the second training set.

Once trained, the recognition algorithm provides the neural network with a current medical intervention data (e.g., patient vital information, patient images, surgical instruments in use, surgical instruments that have been used, surgical instruments that are proximate to the operating table and readily available or use, settings and/or configurations of surgical instruments, location of procedures, actions of supporting medical staff (e.g., anesthesiologist, radiologist, surgical nurse, etc.)) and receiving therefrom the medical intervention situation and, additionally or alternatively, determining a deviation of a current device setting from determined device setting corresponding to the medical intervention situation.

In another embodiment, the intervention situation can also depend on the performing surgeon and/or the surgical team. The surgeon and/or the team can be determined from the corresponding information from an operating room occupancy plan, from the patient's medical record or comparable information. Alternatively or in addition, the operator can be identified via person recognition, in particular via facial recognition. In particular, image data from a camera can be used here, which is recorded in an area of the operating room in which the face is clearly visible, for example where no face mask is yet worn. Alternatively or in addition, the surgeon and/or the surgical team can also be recognized via a corresponding user input via a user interface. Accordingly, for example, personal preferences or needs can be taken into account during configuration or information about the intervention can be derived.

In another embodiment, the necessary adjustments determined can be carried out at least partially automatically or completely automatically (i.e., without human action or intervention). Accordingly, depending on the degree of integration, but also depending on possible direct impacts on the medical intervention or legal requirements, certain changes can be made automatically, while other changes require at user approval. Accordingly, certain adjustments, such as adjustments to lighting, ventilation, air conditioning and the like, may be made automatically, preferably within certain predefined limits. Changes to medical device settings may be protected and cannot be carried out automatically without human authorization.

Accordingly, in another embodiment, the adjustments can be made automatically only after a release is received from a human (e.g., medical staff or other authorized personnel) which then enables automatic application of the changes. Accordingly, adjustments to certain devices, in particular medical devices, or indicia of any adjustments that go beyond certain predefined limits can be displayed or suggested via a corresponding output medium or output device, such as a monitor or a device display. The actual adjustment can then be carried out either manually by the staff or automatically after appropriate approval or confirmation by the staff. The release can be done via a corresponding user input on a user interface or via speech recognition of corresponding verbal instructions.

In another embodiment, deviations can be determined by comparing collected data with data from comparable medical interventions in a corresponding intervention situation based on historical data. In particular, the significance of detected deviations from device settings can be determined using various statistical methods and appropriate adjustments can only be carried out or suggested if the deviation is significant. Corresponding significance values can be predetermined depending on the device and/or settings and stored at the appropriate location. Alternatively or in addition, artificial data from corresponding intervention situations of comparable medical interventions can also be used. In particular, this artificial data may include device settings according to the specifications of the device manufacturers or empirically or theoretically determined preferred device settings for specific applications.

In another embodiment, the determination of deviations can be carried out at least partially on a recognition algorithm based on artificial intelligence. In addition, the recognition algorithm can be or can have been trained with historical data. Here, too, the significance of detected deviations from device settings can be determined using various statistical methods and appropriate adjustments can be made or suggested if the deviation is significant. Corresponding significance values can be predetermined depending on the device and/or settings and stored at the appropriate location. In preferred embodiments, the artificial intelligence-based recognition algorithm can recognize scenarios taking into account all available data, in particular also taking into account sensor data. In particular, based on sensor data, for example acoustic signals, such as communication in the operating room, and the type of movement in the operating room, a stress level can be detected, to which adjustments can be made if necessary. The lighting and/or temperature can be adjusted accordingly.

In a further aspect, a system is disclosed comprising at least one data source in an integrated operating room, at least one configurable device in the integrated operating room, and at least one data processing unit, wherein the system is configured to carry out at least one of the methods described above. Data sources comprise devices and/or sensors that can supply data to the data processing unit. A system may comprise any number of configurable devices and data sources. In preferred embodiments, in addition to the at least one data processing unit and the at least one configurable device, a system may also comprise at least one sensor for detecting various parameters which may be used to detect the intervention situation or to indirectly detect device settings. Corresponding sensors may include brightness sensors, temperature sensors, humidity sensors, cameras, in particular room cameras, microphones and the like.

In another aspect, determining the intervention situation comprises comparing current data with historical data.

In another aspect, the intervention situation also depends on the surgeon performing the intervention.

In another aspect, wherein the determined necessary adjustments are made at least partially automatically.

In another aspect, the adjustments are made automatically after release.

In another aspect, indicia of the determined necessary adjustments are displayed on an output device.

In another aspect, the determination of deviations is carried out at least partly on the basis of a recognition algorithm supported by artificial intelligence.

In another aspect, the recognition algorithm has been trained with historical data.

In another aspect, a system comprising at least one data source in an integrated operating room, at least one configurable device in the integrated operating room, and at least one data processing unit, wherein the system is configured to carry out a method according to one or more aspects herein.

In another aspect, a computer program product comprising commands which, when the program is executed by a computer, cause said computer to perform the steps of the method according to one or more aspects herein.

In another aspect, a computer-readable storage medium comprising commands which, when executed by a computer, cause the computer to carry out the methods according to one or more aspects herein.

In a further aspect, a computer program is disclosed wherein the computer program comprises commands which, when the program is executed by a computer, cause the computer to carry out at least one of the methods described above.

In another aspect, a computer-readable storage medium is disclosed, the storage medium storing commands that, when executed by a computer, cause the computer to perform at least one of the methods described above.

### BRIEF DESCRIPTION OF THE FIGURES

The present disclosure is described in conjunction with the appended figures:

FIG. 1 shows schematically the sequence of the method in accordance with the present disclosure;

FIG. 2 shows schematically the data flow of the method in accordance with according to the present disclosure;

FIG. 3 shows schematically a system in accordance with to the present disclosure; and

FIG. 4 shows an integrated operating room in which the system/method is implemented in accordance with embodiments of the present disclosure.

### DETAILED DESCRIPTION

The ensuing description, including the figures, provide embodiments and aspects of embodiments only and is not intended to limit the scope, applicability, or configuration of the claims. Rather, the ensuing description will provide those skilled in the art with an enabling description for implementing the embodiments. It will be understood that various changes may be made in the function and arrangement of elements without departing from the spirit and scope of the appended claims.

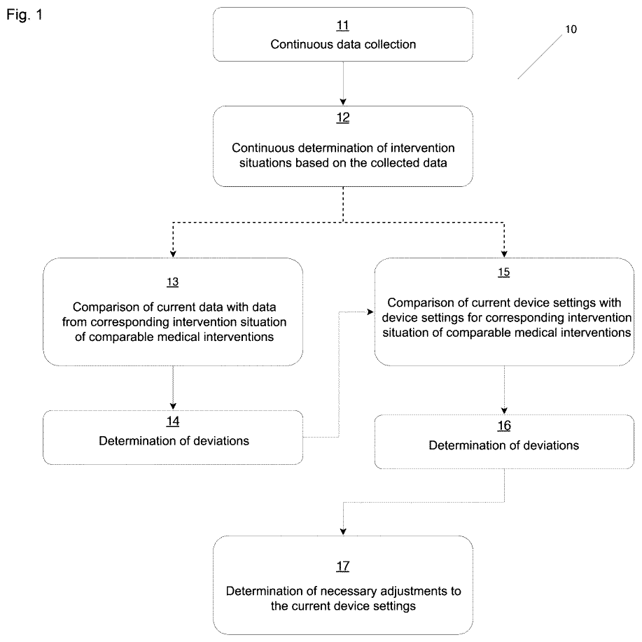

FIG. 1 schematically shows the sequence of the method 10 in accordance with the present disclosure, wherein it should be noted that in some embodiments the sequence of the individual method steps is fundamentally not predetermined and substantially results from the fact that they build on one another at least in part; in particular, some method steps run continuously, so that although results of these method steps can be a prerequisite for a subsequent method step, the respective results do not necessarily conclude or terminate the preceding method step.

In step 11, data is continuously collected from all available data sources. Data can be collected from one or more pure data sources, such as sensors or databases, and/or from one or more configurable devices, which can include device settings, state of a device, values measured by a device, etc.

In step 12, intervention situations are continuously determined based on the collected data. The intervention situation is determined based on the collected data, whereby the collected data may also include corresponding input by the user. In certain embodiments, the intervention situation is determined using algorithms based on artificial intelligence, whereby information concerning the type of medical intervention can be taken into account.

In step 13, the comparison of current data with data from corresponding, particularly historical, intervention situations of comparable medical interventions takes place and, in a subsequent step 14, the determination of deviations between the two data sets. It should be emphasized that the 12, 13 and 14 may affect the entire collected data set or parts of the collected data set, whereby the individual steps may in particular also affect different, possibly overlapping, areas of the data. In particular, the steps of comparing data and identifying deviations can be applied simultaneously or sequentially to different, possibly overlapping, areas of the data. For example, in a first step 13 the data can be compared with data from comparable medical interventions and, in a step 14, if applicable, a deviation from the normal course of a medical intervention, a so-called anomaly, can be detected. In a subsequent step 15, in particular if an anomaly has been detected, individual data areas, preferably the device settings, can be compared with device settings of comparable medical interventions in order to identify possible reasons for the anomaly in the form of deviating device settings in a step 16. Alternatively, without first performing steps 13 and 14, device settings can be compared only in step 15 and then irregularities or deviations can be determined in step 16 solely based on different device settings.

In principle, the inventive determination of deviations between current data, such as device settings, and reference data, such as device settings in a corresponding intervention situation of comparable medical interventions, is carried out according to the principles of anomaly detection. This means that data points in a data set are identified that deviate from the norm, i.e., from data categorized as normal or averaged from corresponding historical intervention situations. Using artificial intelligence-based algorithms, anomaly detection highlights instances that deviate significantly from the usual patterns or statistical models. By detecting deviations/anomalies, potential problems can be automatically identified and, if necessary, resolved.

In anomaly detection, a so-called baseline behavior profile can first be created; according to the certain aspects, the type of medical intervention can be taken into account, which can improve the recognition of patterns. This profile represents the expected patterns and behaviors of the data when the medical intervention proceeds as expected and can be based on reference data accordingly. Historical data, a representative sample of normal behavior, or even simulation data can be used for this purpose. According to the certain aspects, behavioral profiles for various medical interventions, possibly with higher granularity also for intervention situations of the respective medical interventions, can in particular be created in advance and stored in a more suitable location.

The data collected during the medical intervention can be compared—in whole or in part—with one or more corresponding profiles in reference data. In particular, the type of medical intervention, as determined from the collected data or captured from user input, can be taken into account when selecting suitable comparison data. Likewise, aspects, such as the equipment of the operating room, the available data sources, and the composition of the surgical team can also be taken into account when selecting suitable reference data. The data are evaluated and their agreement with the expected characteristics of the normal behavioral profile is measured. All data points that deviate significantly from this are marked as anomalies. These deviations can be identified using various statistical techniques, artificial intelligence-based algorithms or rule-based approaches as known to those skilled in the art.

If the anomalies represent deviations from the intervention situations, i.e., irregularities in the course of the medical intervention, they can be examined more closely in order to determine their causes and, if applicable, their effects. In particular, as described above, deviations at this level can be identified by comparing device settings and, in a step 17, corresponding adjustments to the device settings can be determined and subsequently proposed or implemented.

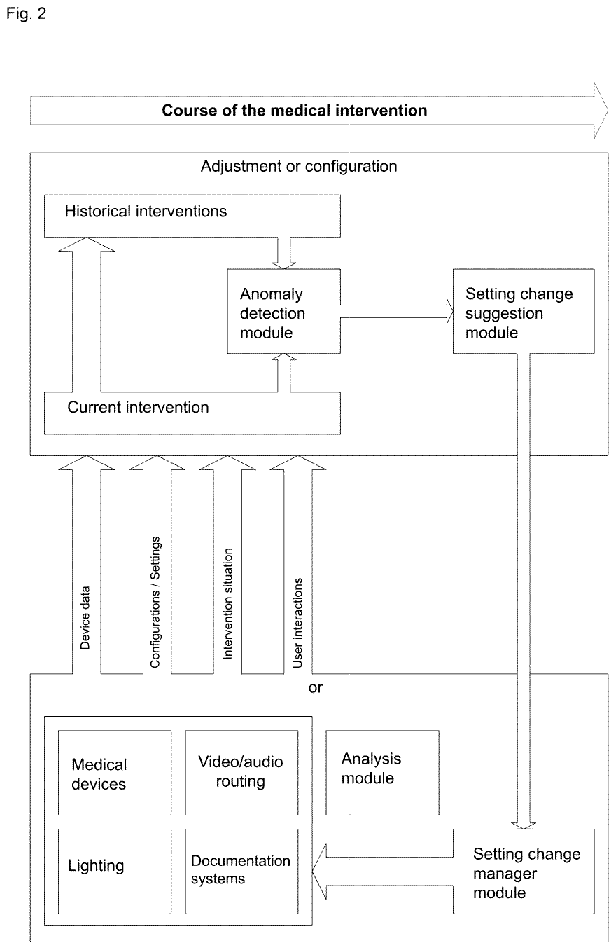

FIG. 2 shows schematically the sequence or data flow of the method in accordance with the present disclosure. Various modules executing the individual process steps are implemented as software modules on one or more computers. During an ongoing medical intervention, various data are continuously collected. As shown in FIG. 2, these data may include various data directly or indirectly related to the intervention. In particular, these data may include device data, i.e., data from medical and non-medical devices integrated into the system, data collected by sensors, configuration and setting information of integrated devices, data relating to the intervention situation, information stored in databases, and data relating to user interactions. According to the certain aspects, all of these data relating to the current intervention are continuously compared by an anomaly detection module with historical data based on the above basic principles of anomaly detection. Alternatively or in addition, simulation data and/or data based on manufacturer specifications can also be used. As described above, deviations/anomalies can be identified using various statistical techniques, artificial intelligence-based algorithms or rule-based approaches as known to those skilled in the art.

If a deviation/anomaly has been detected, a setting change suggestion module can be used to determine suggestions for changes to the device settings by comparing device settings of the current intervention with those of historical interventions and/or manufacturer specifications. These changes to device settings can include, for example, medical device settings, general operating room settings, such as lighting changes, settings affecting video and/or audio routing, and also instructions to a documentation system, as shown in FIG. 2. According to the certain aspects, the suggestions for setting changes are not limited to the functions or devices mentioned above and can accordingly relate to any integrated function and any integrated device.

The setting change suggestions can be passed to a setting change manager module as shown in FIG. 2. Depending on the type of proposed setting change, the setting change manager module can either forward it directly to the corresponding device or implement it directly, or display indicia thereof on a corresponding output device, i.e., suggest it. In the latter case, the proposed setting change can either be made manually by the staff or forwarded to the respective device for automatic implementation by the setting change manager module after approval by the staff. The decision on how to proceed with proposed setting changes, i.e., whether they can be implemented directly or whether they require approval by staff, can be made using an analysis module.

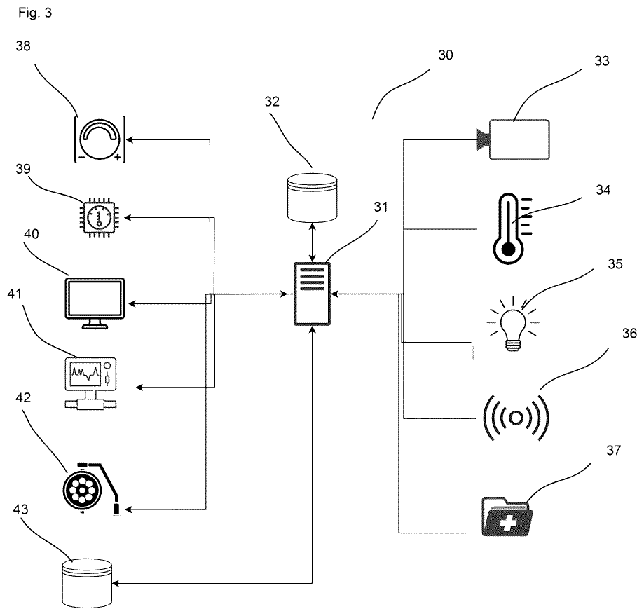

FIG. 3 shows a system 30, in accordance with the present disclosure, for carrying out the method according to certain aspects. The system includes a data processing unit 31 connected to a data memory 32. The data store stores the information to be used for anomaly detection, i.e., reference data, rule sets, training data for artificial intelligence-based algorithms, the trained algorithms, and the like. The system 30 includes various data sources, such as a room camera 33, a temperature sensor 34, a brightness sensor 35, another general sensor 36, and access to digital hospital data 37, such as patient records and/or operating room occupancy plans. The system 30 further includes various configurable devices, such as a brightness controller 38, a temperature controller 39, a monitor 40, a medical device 41, an operating lamp 42 and a documentation system 43. All of the above-mentioned data sources and devices are operationally connected to the data processing unit and thus form the basis of an integrated operating room. It should be noted that the data sources and devices mentioned are merely an example list. It should also be mentioned that, according to certain aspects, the configurable devices themselves also function as data sources, whether they collect data, for example medical data, or whether their respective device settings are read out.

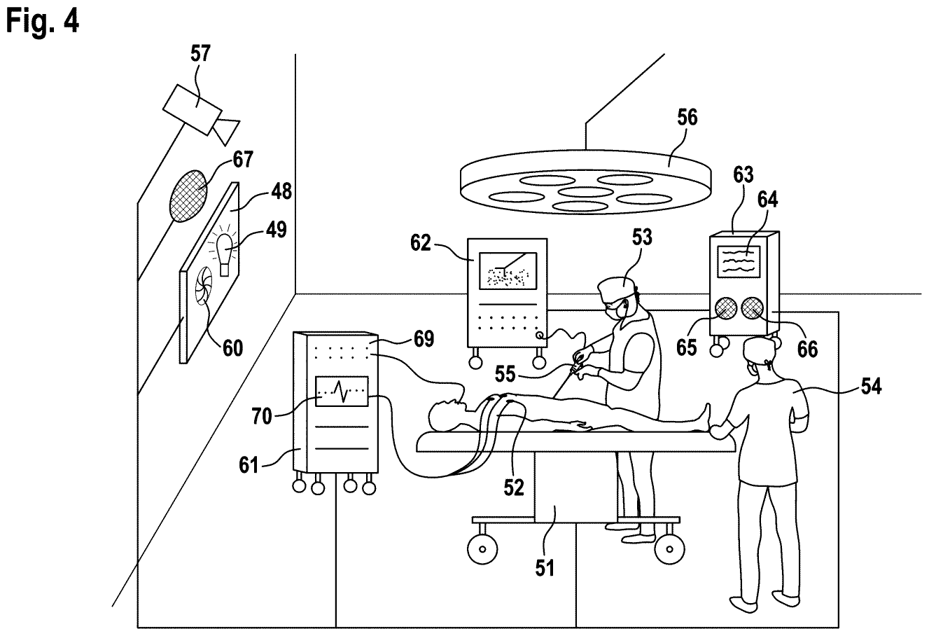

FIG. 4 shows the implementation and application of the method or system, in accordance with the present disclosure, in an integrated operating room in which a minimally invasive intervention is performed as an example. In the operating room in the scenario shown, there is an operating table 51 on which a patient 52 lies. The patient 52 is intubated and is being artificially ventilated by means of a ventilator 69 via a tube 68 inserted into the trachea. The ventilator can be used to monitor and regulate the patient's respiratory function. The patient 52 is also connected to an ECG device via electrodes 70. For simplicity, ventilator 69 and ECG device 70 are shown as a common combination device 61. The operating room further comprises a lighting system 56, a room camera 57, a control unit 48 with elements for lighting control 59 and ventilation control 60 as well as a loudspeaker-microphone combination 67. In the operating room, there is a surgeon 53 and a surgical nurse 54. The surgeon 53 performs a minimally invasive intervention on the patient 52 using an endoscope 55 in the abdominal cavity. The endoscope 45 is connected to a corresponding endoscope control unit 62, with which various functions, such as electrosurgical functions, can be carried out and on which the images taken with the endoscope camera are displayed on a screen. The operating room also contains a control unit 63 comprising a screen 64, a microphone unit 65 and a loudspeaker unit 66, which is configured to carry out the method according to certain aspects. The control unit is connected to all other devices in the operating room, in particular to the operating table 51, the room camera 57, the control unit 58, the combination device 61, the loudspeaker-microphone combination 67 and the endoscope control unit 62, so that device settings and additional information can be collected directly and/or indirectly, as described in detail above. This integration enables—in addition to simplified operation by staff—also central monitoring of the various available devices and, if necessary, even a central, possibly even automated configuration of individual devices.

On the control unit 63, in the example shown, the various modules executing the method according to certain aspects are implemented, although other configurations are also fundamentally encompassed by one or more aspects. Accordingly, the control unit 63 continuously collects data from the connected, integrated devices and sensors. The control unit 63 is operatively connected to a storage unit on which the data to be used for anomaly detection is stored. The storage unit can be integrated into the control unit 63 or configured externally, optionally cloud-based. The data can include historical data from medical interventions, simulated data, but also trained algorithms based on artificial intelligence.

As soon as an irregularity or anomaly is detected by the control unit, possible changes to device settings are determined as described above and either implemented directly or displayed on the output unit 64 and thus suggested to the personnel. For example, if it is detected that the operating lamp 56 is darker than usual for comparable interventions, it can be automatically made brighter. However, if the surgeon performing the intervention has been identified based on the collected data and has previously used lower lighting in his or her interventions, the operating lamp setting can remain unchanged.

Similarly, an irregularity in ventilation can be detected based on the data from the ventilator 69. In this case, the control unit 63 can suggest a change to the settings of the ventilator 69 on the output unit 64 and request approval of the change by the staff. The staff can give the corresponding approval via an input or also via a voice command, whereby the control unit 63 automatically makes the corresponding, proposed change.

In the same way, all collected data can be continuously checked for deviations and/or anomalies and, if necessary, suggestions for changes to device settings can be determined.

According to these two examples, changes to the device settings can be made directly or indirectly on all adjustable integrated devices.

The scope of this disclosure includes all changes, substitutions, variations, alterations, and modifications to the exemplary embodiments described or illustrated herein that would be obvious to a person skilled in the art. The scope of this disclosure is not limited to the embodiments described or illustrated herein. In addition, although this disclosure describes and illustrates certain embodiments herein as encompassing particular components, elements, features, functions, operations, or steps, any of these embodiments may also include any combinations or permutations of any components, elements, features, functions, operations, or steps that would be obvious to a person skilled in the art. A reference in the appended claims to a method or apparatus or component of an apparatus or system being adapted, arranged, capable, configured, enabled, operable or ready to perform a particular function additionally includes this apparatus, system or component, regardless of whether it or this particular function is activated, turned on or enabled, as long as this apparatus, system or component is adapted, arranged, capable, configured, enabled, operable or ready to perform it. Although this disclosure describes or illustrates certain embodiments as being advantageous, certain embodiments may also provide none, some, or all of these advantages.

What is claimed is:

1. A method of dynamically configuring an integrated operating room, comprising:
collecting data corresponding to an ongoing medical intervention comprising one or more of a device setting, a device display, a device performance, a user, a user action, and information describing the ongoing medical intervention;
determining an intervention situation based on the collected data;
comparing a current device setting of the device with a comparable device setting corresponding to a comparable intervention situation of a comparable medical intervention;
determining a deviation of the current device setting from the comparable device setting in the comparable intervention situation of a comparable medical intervention; and
determining an adjustment to the current device setting to attenuate the deviation.

2. The method of claim 1, wherein:
collecting data corresponding to an ongoing medical intervention further comprises continuously collecting data corresponding to the ongoing medical intervention;
determining intervention situation comprises continuously determining the intervention situation; and
comparing the current device setting with the comparable device setting comprises continuously comparing the current device setting with the comparable device setting.

3. The method of claim 1, wherein determining the intervention situation comprises comparing the current data with historical data of one or more comparable medical interventions.

4. The method of claim 1, wherein determining the intervention situation is further comprises determining the intervention situation in accordance with an attribute of a particular surgeon performing the medical intervention.

5. The method of claim 1, further comprising, automatically applying the determined necessary adjustment to the current.

6. The method of claim 5, wherein the determined necessary adjustment automatically applied upon receiving a release.

7. The method of claim 1, wherein indicia of the determined necessary adjustment are displayed on an output device.

8. The method of claim 1, wherein the determination of deviation is carried out at least partly on the basis of a recognition algorithm utilizing artificial intelligence.

9. The method of claim 8, wherein the recognition algorithm has been trained with historical data.

10. A system, comprising:
a device utilized during an ongoing medical intervention; and
a computer comprising a processor coupled to a non-transitory memory comprising instructions to cause the computer to perform:
collecting data, corresponding to an ongoing medical intervention, comprising one or more of a device setting, a device display, a device performance, a user, a user action, and information describing the ongoing medical intervention;
determining an intervention situation based on the collected data;
comparing a current device setting of the device with a comparable device setting corresponding to a comparable intervention situation of a comparable medical intervention;
determining a deviation of the current device setting from the comparable device setting in the comparable intervention situation of a comparable medical intervention; and
determining an adjustment to the current device setting to attenuate the deviation.

11. The system of claim 10, wherein:
collecting data corresponding to an ongoing medical intervention further comprises continuously collecting data corresponding to the ongoing medical intervention;
determining intervention situation comprises continuously determining the intervention situation; and
comparing the current device setting with the comparable device setting comprises continuously comparing the current device setting with the comparable device setting.

12. The system of claim 10, wherein determining the intervention situation comprises comparing the current data with historical data of one or more comparable medical interventions.

13. The system of claim 10, wherein determining the intervention situation is further comprises determining the intervention situation in accordance with an attribute of a particular surgeon performing the medical intervention.

14. The system of claim 10, further comprising, automatically applying the determined necessary adjustment to the current.

15. The system of claim 14, wherein the determined necessary adjustment automatically applied upon receiving a release.

16. The system of claim 10, wherein indicia of the determined necessary adjustment are displayed on an output device.

17. The system of claim 10, wherein the determination of deviation is carried out at least partly on the basis of a recognition algorithm utilizing artificial intelligence.

18. The system of claim 17, wherein the recognition algorithm has been trained with historical data.

19. A computer-readable medium comprising non-transitory instructions that, when read by a machine, cause the machine to perform:
collecting data, corresponding to an ongoing medical intervention, comprising one or more of a device setting, a device display, a device performance, a user, a user action, and information describing the ongoing medical intervention;
determining an intervention situation based on the collected data;
comparing a current device setting of the device with a comparable device setting corresponding to a comparable intervention situation of a comparable medical intervention;
determining a deviation of the current device setting from the comparable device setting in the comparable intervention situation of a comparable medical intervention; and
determining an adjustment to the current device setting to attenuate the deviation.

20. The computer-readable medium of claim 19, wherein:
collecting data corresponding to an ongoing medical intervention further comprises continuously collecting data corresponding to the ongoing medical intervention;
determining intervention situation comprises continuously determining the intervention situation; and
comparing the current device setting with the comparable device setting comprises continuously comparing the current device setting with the comparable device setting.
